(19) United States
(12) Patent Application Publication
(10) Pub. No.: US 2024/0387001 A1
(43) Pub. Date: Nov. 21, 2024

(54) MACHINE LEARNING-BASED PREDICTION OF BIOLOGICAL CONSTITUENTS IN A SAMPLE

(71) Applicant: ILLUMINA, INC., San Diego, CA (US)

(72) Inventors: Steven Flygare, Salt Lake City, UT (US); Robert Schlaberg, Salt Lake City, UT (US); Kate Mariel Broadbent, Salt Lake City, UT (US)

(21) Appl. No.: 18/662,819

(22) Filed: May 13, 2024

Related U.S. Application Data
(60) Provisional application No. 63/502,393, filed on May 15, 2023.
(60) Provisional application No. 63/505,366, filed on May 31, 2023.

(51) Int. Cl. G16B 40/00; G16B 20/00

(52) U.S. Cl. CPC G16B 40/00; G16B 20/00

(57) ABSTRACT

Methods and systems that include training a machine learning model for detecting biological constituents in a sample are provided. A computer-implemented method, and systems executing the method, may include collecting metagenomic data that includes biological constituents obtained from a sample; generating a first molecular data set of covariates; generating a second molecular data set of covariates; generating a training set comprising the first and second covariates as well as combined covariates; and training the machine learning model using aggregate molecular covariates to predict biological constituents from metagenomics data.

### INCORPORATION BY REFERENCE TO ANY PRIORITY APPLICATIONS

This application claims priority to U.S. Provisional Application No. 63/502,393 filed May 15, 2023 and U.S. Provisional Application No. 63/505,366 filed May 31, 2023, the content of each of which is incorporated by reference in its entirety. Any and all applications for which a foreign or domestic priority claim is identified in the Application Data Sheet as filed with the present application are hereby incorporated by reference under 37 CFR 1.57.

### BACKGROUND

Metagenomics, the genomic analysis of a population of microbes, makes possible the profiling of microbial communities in the environment and the human body at unprecedented depth and breadth. Its rapidly expanding use is revolutionizing our understanding of microbial diversity in natural and man-made environments and is linking microbial community profiles with health and disease. To date, most studies have relied on PCR amplification of microbial marker genes (e.g., bacterial 16S rRNA), for which large, curated databases have been established. More recently, higher throughput and lower cost sequencing technologies have enabled a shift towards direct from specimen metagenomic approaches (both targeted and target-independent). These approaches can reduce bias as they do not involve PCR primer binding, improve the resolution of genetically related taxa, and enable discovery of novel pathogens.

While conventional, pathogen-specific nucleic acid amplification tests are highly sensitive and specific, they often require a priori knowledge of likely pathogens. The result is increasingly large, yet inherently limited diagnostic panels to enable diagnosis of the most common pathogens. In contrast, target-independent high-throughput sequencing allows for unbiased, hypothesis-free detection and molecular typing of a theoretically unlimited number of common and unusual pathogens. Wide availability of next-generation sequencing instruments, lower reagent costs, and streamlined sample preparation protocols are enabling an increasing number of investigators to perform high-throughput DNA and RNA-seq for metagenomics studies. However, analysis of sequencing data is still difficult and time-consuming, requiring bioinformatics skills, computational resources, and microbiological expertise that is not available to many laboratories, especially diagnostic ones.

### SUMMARY

The methods and systems disclosed herein each have several aspects, no single one of which is solely responsible for their desirable attributes. Without limiting the scope of the claims, some prominent features will now be discussed briefly. Numerous other embodiments are also contemplated, including embodiments that have fewer, additional, and/or different components, steps, features, objects, benefits, and advantages. The components, aspects, and steps may also be arranged and ordered differently. After considering this discussion, and particularly after reading the section entitled “Detailed Description”, one will understand how the features of the devices and methods disclosed herein provide advantages over other known devices and methods.

A computer-implemented method of training a machine learning model for detecting biological constituents in a sample, the method including collecting metagenomic data from biological constituents in a sample; generating a first molecular data set; generating a second molecular data set; creating a training set comprising an aggregated set of the first and second molecular data sets; and training the machine learning model using the training set. In some embodiments, the machine learning model comprises a random forest model. In some embodiments, the machine learning model comprises a deep neural network (DNN). In some embodiments, the machine learning model comprises a convolutional neural network (CNN). In some embodiments, the machine learning model comprises a support vector machine (SVM). In some embodiments, the machine learning model comprises a categorical classifier.

In some embodiments, the method further comprises selecting a first machine learning model based on one or more metrics of the first molecular data set. In some embodiments, the method further comprises selecting a second machine learning model based on one or more metrics of the second molecular data set. In some embodiments, the first and second machine learning models are the same. In some embodiments, the first and second machine learning models are different.

In some embodiments, the generating of the first molecular data set comprises applying an aligner-based classifier to the collected metagenomic data against a first source, and the generating of the second molecular data set comprises applying the aligner-based classifier to the collected metagenomic data against a second source. In some embodiments, the generating of the first molecular data set comprises applying a de novo assembler to the collected metagenomic data against a first source, and the generating of the second molecular data set comprises applying the de novo assembler to the collected metagenomic data against a second source. In some embodiments, the generating of the first molecular data set comprises applying a k-mer based classifier to the collected metagenomic data against a first source, and the generating of the second molecular data set comprises applying the k-mer based classifier to the collected metagenomic data against a second source. In some embodiments, the generating of the first molecular data set comprises applying a classifier to the collected metagenomic data against a first source, and the generating of the second molecular data set comprises applying the classifier to the collected metagenomic data against a second source. In some embodiments, the classifier comprises a categorical classifier. In some embodiments, the classifier comprises a k-mer based classifier. In some embodiments, the first source comprises a first database. In some embodiments, the second source comprises a second database.

In some embodiments, the first and second molecular data sets comprise polypeptides. In some embodiments, the first and second molecular data sets comprise polynucleotides. In some embodiments, the first source comprises a curated set of polynucleotides. In some embodiments, the curated set of polynucleotides comprises one or more genomes. In some embodiments, the polynucleotides of the second molecular data set comprise publicly available polynucleotides. In some embodiments, the publicly available polynucleotides comprise one or more publicly available genomes. In some embodiments, the first source comprises a curated set of polypeptides. In some embodiments, the curated set of the polypeptides comprises one or more proteomes. In some embodiments, the second source comprises publicly available polypeptides. In some embodiments, the publicly available polypeptides comprise one or more publicly available proteomes.

In some embodiments, the first and second molecular data sets comprise a plurality of taxids. In some embodiments, the method further comprises aggregating the first and second molecular data sets for each of the taxids. In some embodiments, the k-mer based classifier comprises taxonomer. In some embodiments, the k-mer based classifier comprises KRAKEN.

In some embodiments, the method further comprises detecting, from an output of the machine learning model using the training set, a presence of one or more of the biological constituents obtained from the sample based on a probability value. In some embodiments, the method further comprises detecting, from an output of the machine learning model using the training set, an absence of one or more of the biological constituents obtained from the sample. In some embodiments, the sample is sourced from one or more environmental sources, one or more industrial sources, one or more subjects, one or more populations of microbes, or a combination thereof. In some embodiments, the polynucleotides obtained from the sample includes one or more polynucleotides from one or more pathogens. In some embodiments, the generating of the first and second molecular data sets occurs in parallel. In some embodiments, the method further comprises iterating the first molecular data set. In some embodiments, the method further comprises iterating the second molecular data set.

In some embodiments, a system for detecting biological constituents in a sample is provided, the system including one or more processors that are programmed to execute a method that includes obtaining metagenomic data, wherein the metagenomic data is obtained from biological constituents in a sample; generating a first molecular data set; generating a second molecular data set; creating a training set comprising an aggregated set of the first and molecular data sets; and training the machine learning model using the training set.

In some embodiments, the machine learning model comprises a random forest model. In some embodiments, the machine learning model comprises a deep neural network (DNN). In some embodiments, the machine learning model comprises a convolutional neural network (CNN). In some embodiments, the machine learning model comprises a support vector machine (SVM). In some embodiments, the machine learning model comprises a categorical classifier.

In some embodiments, the one or more processors are further programmed to execute a method including selecting a first machine learning model based on one or more metrics of the first molecular data set. In some embodiments, the one or more processors are further programmed to execute a method comprising selecting a second machine learning model based on one or more metrics of the second molecular data set. In some embodiments, the first and second machine learning models are the same. In some embodiments, the first and second machine learning models are different.

In some embodiments, the generating of the first molecular data set comprises applying an aligner-based classifier to the collected metagenomic data against a first source, and the generating of the second molecular data set comprises applying the aligner-based classifier to the collected metagenomic data against a second source. In some embodiments, the generating of the first molecular data set comprises applying a de novo assembler to the collected metagenomic data against a first source, and the generating of the second molecular data set comprises applying the de novo assembler to the collected metagenomic data against a second source. In some embodiments, the generating of the first molecular data set comprises applying an k-mer based classifier to the collected metagenomic data against a first source, the generating of the second molecular data set comprises applying the k-mer based classifier to the collected metagenomic data against a second source. In some embodiments, the generating of the first molecular data set comprises applying a classifier to the collected metagenomic data against a first source, and the generating of the second molecular data set comprises applying the classifier to the collected metagenomic data against a second source. In some embodiments, the classifier comprises a categorical classifier. In some embodiments, the classifier comprises a k-mer based classifier. In some embodiments, the first source comprises a first database.

In some embodiments, the second source comprises a second database. In some embodiments, the first and second molecular data sets comprise polypeptides. In some embodiments, the first and second molecular data sets comprise polynucleotides. In some embodiments, the first source comprises a curated set of polynucleotides. In some embodiments, the curated set of polynucleotides comprises one or more genomes. In some embodiments, the polynucleotides of the second molecular data set comprise publicly available polynucleotides. In some embodiments, the publicly available polynucleotides comprise one or more publicly available genomes. In some embodiments, the first source comprises a curated set of polypeptides. In some embodiments, the curated set of the polypeptides comprises one or more proteomes. In some embodiments, the second source comprises publicly available polypeptides. In some embodiments, the publicly available polypeptides comprise one or more publicly available proteomes. In some embodiments, the first and second molecular data sets comprise a plurality of taxids. In some embodiments, the one or more processors are further programmed to execute a method comprising aggregating the first and second molecular data sets for each of the taxids. In some embodiments, the k-mer based classifier comprises taxonomer. In some embodiments, the k-mer based classifier comprises KRAKEN.

In some embodiments, the one or more processors are further programmed to execute a method comprising detecting, from an output of the machine learning model using the training set, a presence of one or more of the biological constituents obtained from the sample based on a probability value. In some embodiments, the one or more processors are further programmed to execute a method comprising detecting, from an output of the machine learning model using the training set, an absence of one or more of the biological constituents obtained from the sample. In some embodiments, the sample is sourced from one or more environmental sources, one or more industrial sources, one or more subjects, one or more populations of microbes, or a combination thereof. In some embodiments, the polynucleotides obtained from the sample includes one or more polynucleotides from one or more pathogens. In some embodiments, the generating of the first and second molecular data sets occurs in parallel. In some embodiments, the one or more processors are further programmed to execute a method comprising iterating the generating of the first molecular data set. In some embodiments, the one or more processors are further programmed to execute a method comprising iterating the generating of the second molecular data set.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “figure” and “FIG.” herein), of which:

FIGS. 1A-B illustrate standard metagenomics pipeline metrics for 2 different pathogens. FIG. 1A illustrates coverage data for the genome of Klebsiella pneumoniae in samples where K. pneumoniae was expected (1) and where it was not expected (0). FIG. 1B illustrates read count data for the genome of Escherichia coli in samples where E. coli was expected (1) and where it was not expected (0).

FIG. 2A is a flowchart diagram illustrating a workflow to train machine learning (ML) models according to some embodiments. FIG. 2B is a flowchart diagram illustrating a workflow to train machine learning (ML) models according to some embodiments.

FIG. 3A is a flowchart diagram illustrating a workflow of collecting metagenomic data from a sample and training ML models that includes iteration according to some embodiments. FIG. 3B is a flowchart diagram illustrating a workflow of collecting metagenomic data from a sample and training ML models that includes iteration according to some embodiments.

FIG. 4A is a flowchart diagram illustrating a workflow of collecting metagenomic data from a sample and training ML models according to some embodiments. FIG. 4B is a flowchart diagram illustrating a workflow of collecting metagenomic data from a sample and training ML models according to some embodiments.

FIG. 5 is a flowchart diagram illustrating a workflow to train the ML models according to some embodiments.

FIG. 6 illustrates an F-beta score comparison between a production metagenomics pipeline (x-axis) to a metagenomics pipeline incorporating the trained ML model (y-axis) of FIG. 5.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Accurately detecting biological constituents from samples of diverse origins is challenging. Depending on the sample origin, metagenomics pipelines can produce dozens of identifications from a single biological sample. The sheer amount of detection can be overwhelming and difficult to interpret. Additionally, it can be difficult to know which detected biological constituents are true or false positives. A non-exhaustive list of reasons for this uncertainty includes reference sequence misannotations, high genetic similarity between biological references, incomplete reference databases, e.g., query sequence is not directly represented, and sample artifacts. Standard metagenomics pipelines often rely on individual metrics, such as genome coverage or read count to filter detections. Using individual metrics with set thresholds is often insufficient to reliably detect many important biological constituents. Even using multiple metrics together with set thresholds may be unable to adapt to normal sample-to-sample variability (e.g., changing organism composition; changing sample type, etc.). Therefore, state of the art metagenomics data analysis using known pipelines lack specificity and sensitivity (unsatisfactory levels of false positives and negatives), lack scalability, and are often time intensive. To overcome the shortcomings of current threshold-based approaches, various embodiments of systems and methods that train models to utilize machine learning are provided herein to better distinguish between signal and noise in metagenomics samples (including, but not limited to, targeted and target-independent metagenomics) to improve biological constituent detection/identification accuracy. With this partial or fully automated approach, the systems and methods can scale to process thousands of samples per day versus the 20-50 samples a day processed by a trained artisan. In a non-limiting example, the systems and methods disclosed herein are aimed to detect an element molecularly using polypeptides or nucleic acids where background noise is competing with a signal of interest. Non-limiting examples include “shotgun” metagenomics, enrichment sequencing, and amplicon sequencing. In any of the embodiments described herein, other types of pipelines may be contemplated (e.g., metatranscriptomics, metaproteomics, metabolomics, or the like) in place of the metagenomics pipelines. In some embodiments, the one or more other types of pipelines may be combined with one or more of the metagenomics pipelines in a multinomics/meta-omics approach.

As used herein, the term “biological constituents” refers to any molecular or cellular component, part, substance, or entity that exists within, is produced by, or constitutes a biological organism. Such constituents can encompass, but are not limited to, cells, cellular substructures (e.g., organelles and the like), polypeptides, proteins, enzymes, nucleic acids/polynucleotides (e.g., DNA and RNA), genes, lipids, carbohydrates, hormones, neurotransmitters, metabolites, and other bioactive or structural molecules. In particular, “biological constituents” may be isolated or derived from an organism, being found in tissues, organs, blood, or other bodily fluids. It should be understood that “biological constituents” may occur in their natural state, be synthetically derived, or engineered via genetic, biochemical, or other methods, yet still maintain their essential functional or structural characteristics. The term, “biological constituents,” also encompasses those components found in the context of, or constitutes, microbial organisms, such as bacteria, viruses, fungi, and protozoa. These may include bacterial cell walls, viral capsids, fungal spores, protozoan cysts, or the genetic material contained within these entities. The term, “biological constituents,” also encompasses components that form part of an organism's virulence characteristics or antimicrobial resistance, such as virulence factors and antimicrobial resistant markers. As used herein, “virulence factors” include but are not limited to proteins, toxins, or other molecules that enhance the ability of a pathogen to infect and cause disease in a host organism. As used herein, “antimicrobial resistant markers” (AMR) refer to genes, proteins, or other molecular structures or mechanisms that confer resistance to antimicrobial agents, such as antibiotics, antifungals, or antivirals. Moreover, “biological constituents” can refer to those molecules or structures, or their9erivateives, which form part of the organism's responses to internal or external stimuli. These include, but are not limited to, antibodies, antigens, signaling molecules, and other components of the immune, endocrine, or nervous systems. The term is not limited to living or viable constituents and may also refer to components that have been inactivated, degraded, or otherwise modified while retaining some biological, structural, or functional characteristics of relevance. In some embodiments, the detection and identification of biological constituents includes confirming the presence of a biological entity (taxon) or characterizing genomic markers for specific phenotypes, such as drug resistance, pathogenicity, specific strains/variants/genotypes, and any combinations thereof.

As used herein, the term “pathogen” refers to any biological entity or constituent, as previously defined, capable of causing disease, illness, or abnormality in a susceptible host organism. The term “pathogen” encompasses organisms, such as bacteria, viruses, fungi, and protozoa, as well as prions and other entities, which upon exposure to a susceptible host, can lead to symptomatic or asymptomatic infection. Pathogens may possess and express one or more virulence factors, including, but not limited to, toxins, surface proteins, enzymes, and other molecules, that enhance their ability to infect and cause disease in a host organism. Furthermore, pathogens can harbor antimicrobial resistance markers that render them resistant to one or more antimicrobial agents, making the infection difficult to treat with standard therapies. Pathogens may be naturally occurring, artificially created, or genetically engineered, and can interact with the host's immune system to cause an immune response. They can exist in various forms, including but not limited to, spores, cysts, free-living, intracellular, or extracellular forms. They can be present in various environments, such as water, soil, air, and within living organisms, and can be transmitted by numerous routes, including but not limited to, airborne, direct contact, indirect contact, vector-borne, foodborne, and waterborne routes. The term “pathogen” also includes those entities that, while not typically causing disease in a healthy host organism, can become pathogenic under certain circumstances, such as in hosts with compromised immune systems, a phenomenon known as opportunistic infection.

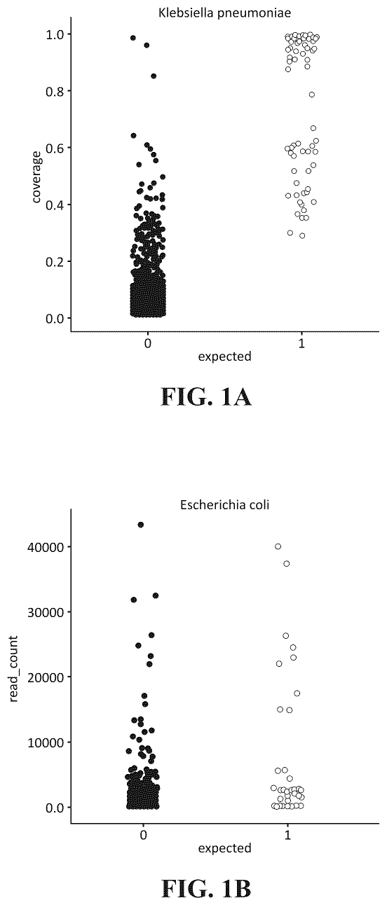

With reference to FIGS. 1A-B, K. pneumoniae and E. coli are pathogens with a high impact on human health. FIG. 1A shows coverage of the K. pneumoniae genome in data sets that were expected to have K. pneumoniae and data sets that were not expected to have K. pneumoniae. As shown, there is an overlap between the expected and non-expected groups, so it will not be possible to use the coverage threshold to accurately distinguish between true and false coverage signals. FIG. 1B shows the coverage of the E. coli genome in data sets that were expected to have E. coli and data sets that were not expected to have E. coli. Unlike in FIG. 1A, the metric in FIG. 1B is read counts classified to E. coli genomes in data sets that were expected to have E. coli and data sets that were not expected to have E. coli. However, the results shown in FIG. 1B indicate a degree of overlap between the classified read counts that makes it impossible to decide on a single read count threshold that would accurately determine when E. coli is present.

For many organisms that are important to human health, a single threshold is insufficient to provide an accurate determination whether an organism of interest is present or not in a sample. Standard metagenomics pipelines attempt to deploy single thresholds to the detriment of sensitivity/specificity. Additional efforts are directed to creating an analytical schema composed of multiple metrics and thresholds based on reference databases and read content. Examples include reads per kilobase per million reads mapped (RPKM) computed in the Explify pipeline (Almas S, et al. Deciphering Microbiota of Acute Upper Respiratory Infections: A Comparative Analysis of PCR and mNGS Methods for Lower Respiratory Trafficking Potential. Adv. Respir. Med., 91, 49-65 (2023)) and the Braken statistic computed in the KRAKEN metagenomics pipeline (Lu J, et al., Bracken: estimating species abundance in metagenomics data. PecrJ Comput. Sci. 3, e104 (2017)). While these schemas are a step forward from simple genome coverage or read count thresholds, they are still inadequate to account for the amount and diversity of noise in metagenomics data.

As used herein, “k-mer” refers to the subsequences of a given length k that make up a sequencing read. For example, the sequence “AGCTCT” can be divided into the 3-nt subsequences “AGC,” “GCT,” “CTC,” and “TCT.” In this example, each of these subsequences is a k-mer, wherein k=3. K-mers may be overlapping or non-overlapping.

Sequence comparison may include one or more comparison steps in which one or more k-mers of a sequencing read are compared to k-mers of one or more reference sequences (also referred to simply as a “reference”). In some embodiments, a k-mer is about or more than about 3 nt, 4 nt, 5 nt, 6 nt, 7 nt, 8 nt, 9 nt, 10 nt, 11 nt, 12 nt, 13 nt, 14 nt, 15 nt, 16 nt, 17 nt, 18 nt, 19 nt, 20 nt, 25 nt, 30 nt, 35 nt, 40 nt, 45 nt, 50 nt, 75 nt, 100 nt, or more in length. In some embodiments, a k-mer is about or less than about 30 nt, 25 nt, 20 nt, 15 nt, 10 nt, or fewer in length. The k-mer may be in the range of 3 nt to 13 nt, 5 nt to 25 nt in length, 7 nt to 99 nt, or 3 nt to 99 nt in length. The length of k-mer analyzed at each step may vary. For example, a first comparison may compare k-mers in a sequencing read and a reference sequence that are 21 nt in length, whereas a second comparison may compare k-mers in a sequencing read and a reference sequence that are 7 nt in length. For any given sequence in a comparison step, k-mers analyzed may be overlapping (such as in a sliding window), and may be of same or different lengths. While k-mers are generally referred to herein as nucleic acid sequences, sequence comparison also encompasses comparison of polypeptide sequences, including comparison of k-mers consisting of amino acids.

Machine learning provides a flexible framework for pattern-learning in complex data to build predictive models that can account for multiple covariates, which includes models that produce binary predications to model an organism being present or not present in a sample. Several models were trained with metagenomics data to assess their relative performance with various molecular data sets. These models include perceptrons, logistic regression, random forests (RF), deep neural networks (DNNs), and convolutional neural networks (CNNs). Machine learning utilizing RF models generalized well and provided accurate predictions, given the available evidence, in determining whether an organism is present or not in a sample.

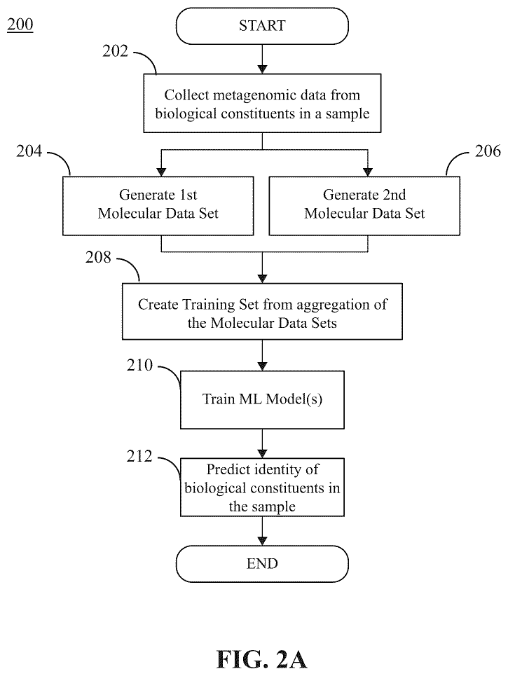

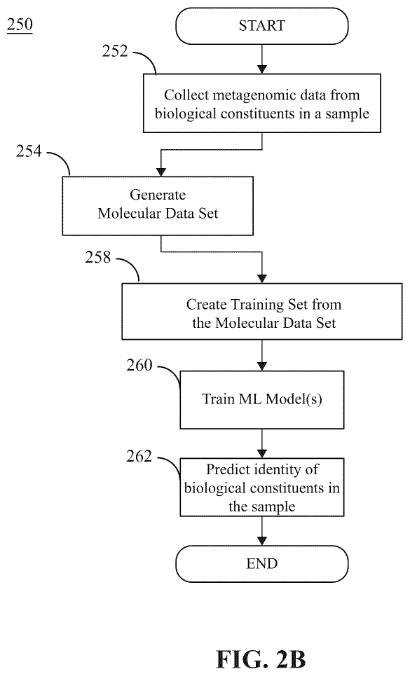

FIG. 2A shows one embodiment of a computer-implemented method 200 of training a machine learning model to predict the identity of biological constituents in a sample. The method 200 begins at a start block and then includes collecting metagenomic data from biological constituents in a sample as shown in block 202. The method further includes generating a first molecular data set as shown in block 204, and generating a second molecular data set as shown in block 206. In some embodiments, the number of molecular data sets is greater than two. Once the molecular data sets have been created, the method 200 further includes creating a training set from aggregation of the first and second molecular data sets as shown in block 208.

In some embodiments, the number of molecular data sets is 3, 4, 5, 6, 7, 8, 9, 10, 50, 100, 200, 500, 1000, 2500, 5000, 10000, 50000, 100000, 500000, 1000000, or more, or a number in a range defined by any two of the preceding values. Once the training set has been created, the method 200 further includes training a machine learning model as shown in block 210 using the training set. Based on the output provided by the trained ML model, a prediction of an identity for the biological constituents from the sample is made at block 212. In some embodiments, based on the output provided by the trained ML model, a prediction of a presence of particular biological constituents found in the sample is provided. In some embodiments, based on the output provided by the trained ML model, a prediction of an absence of particular biological constituents from the sample is provided. In some embodiments, the first and second molecular data sets are produced iteratively, which further updates the training set thereby continuing to train the ML model(s).

FIG. 2B shows another embodiment of a computer-implemented method 250 of training a machine learning model to predict the identity of biological constituents in a sample. The method 250 begins at a start block and then includes collecting metagenomic data from biological constituents in a sample as shown in block 252. The method further includes generating a molecular data set as shown in block 254. Once the molecular data set has been created, the method 250 further includes creating a training set from the molecular data set as shown in block 258.

Once the training set has been created, the method 250 further includes training a machine learning model as shown in block 260 using the training set. Based on the output provided by the trained ML model, a prediction of an identity for the biological constituents from the sample is made at block 262. In some embodiments, based on the output provided by the trained ML model, a prediction of a presence of particular biological constituents found in the sample is provided. In some embodiments, based on the output provided by the trained ML model, a prediction of an absence of particular biological constituents from the sample is provided. In some embodiments, the molecular data set is produced iteratively, which further updates the training set thereby continuing to train the ML model(s).

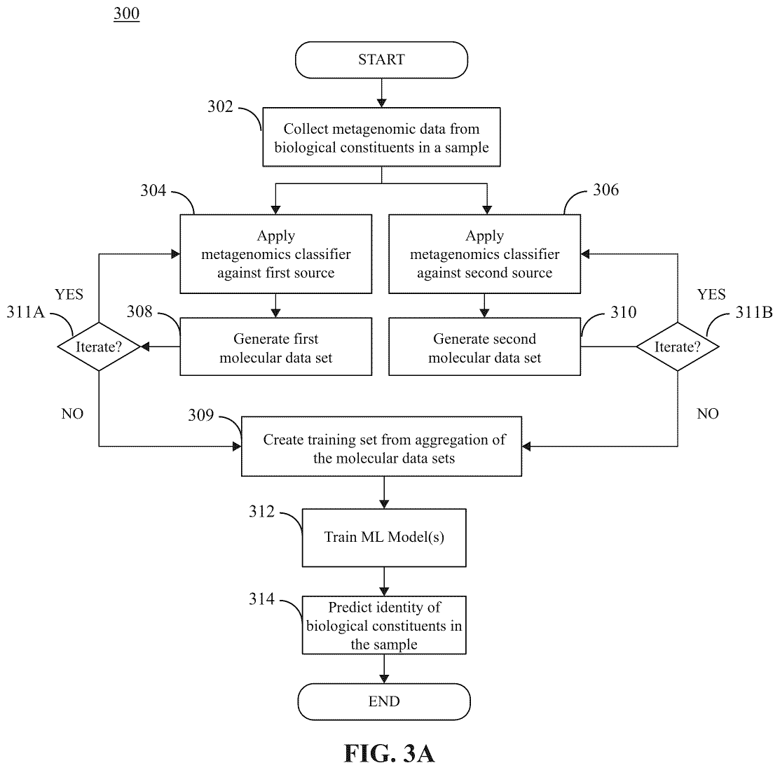

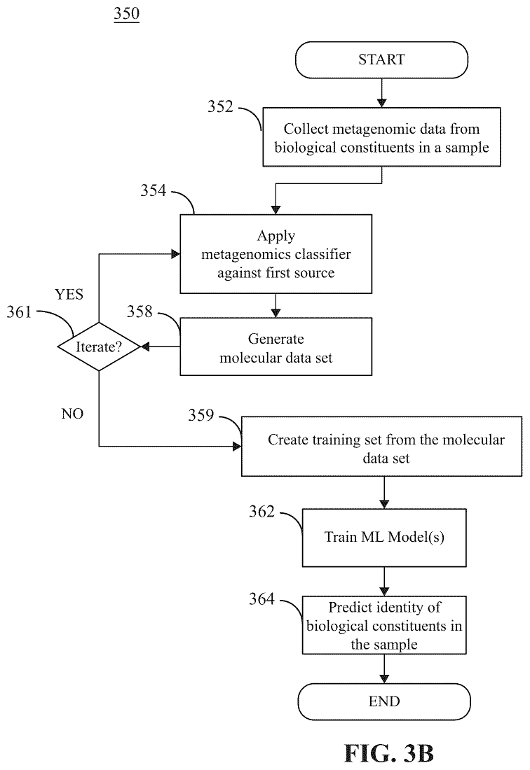

FIG. 3A shows an alternative computer-implemented method 300 of training a machine learning model for detecting biological constituents in a sample. The method 300 begins at a start state and then the method 300 includes collecting metagenomic data from biological constituents in a sample as shown in block 302. The method 300 then applies a metagenomics classifier against a first source at a block 304 and then generates a first molecular data set as shown in block 308. The method 300 also applies a metagenomics classifier against a second source at a block 306 and generates a second molecular data set as shown in block 310. Once the first molecular data set has been created at the block 308 the method moves to a decision state 311A to determine if the method should iterate. If the method should iterate then the method 300 returns to the block 304. If the method should not iterate, then the method 300 moves to the block 309 to create a training set from the aggregation of the molecular data sets. Similarly, once the second molecular data set has been created at the block 310 the method moves to a decision state 311B to determine if the method should iterate. If the method should iterate then the method 300 returns to the block 306. If the method should not iterate then the method 300 moves to the block 309 to create the training set from the aggregation of the molecule data sets.

In some embodiments, the number of molecular data sets is greater than two. Once the training sets have been created, the method 300 moves to block 312 where the ML models can be trained. In some embodiments, the number of molecular data sets is 3, 4, 5, 6, 7, 8, 9, 10, 50, 100, 200, 500, 1000, 2500, 5000, 10000, 50000, 100000, 500000, 1000000, or more, or a number in a range defined by any two of the preceding values. Based on the output provided by the trained ML model a prediction of an identity for the biological constituents from the sample is made at block 314. In some embodiments, based on the output provided by the trained ML model, a prediction of a presence of the biological constituents from the sample is provided. In some embodiments, based on the output provided by the trained ML model, a prediction of an absence of the biological constituents from the sample is provided.

FIG. 3B shows another alternative computer-implemented method 350 of training a machine learning model for detecting biological constituents in a sample. The method 350 begins at a start state and then the method 350 includes collecting metagenomic data from biological constituents in a sample as shown in block 352. The method 350 then applies a metagenomics classifier against a source at a block 354 and then generates a molecular data set as shown in block 358. Once the molecular data set has been created at the block 358 the method moves to a decision state 361 to determine if the method should iterate. If the method should iterate then the method 350 returns to the block 354. If the method should not iterate, then the method 350 moves to the block 359 to create a training set from the molecular data set.

Once the training set or sets have been created, the method 350 moves to block 362 where the ML models can be trained. Based on the output provided by the trained ML model a prediction of an identity for the biological constituents from the sample is made at block 364. In some embodiments, based on the output provided by the trained ML model, a prediction of a presence of the biological constituents from the sample is provided. In some embodiments, based on the output provided by the trained ML model, a prediction of an absence of the biological constituents from the sample is provided.

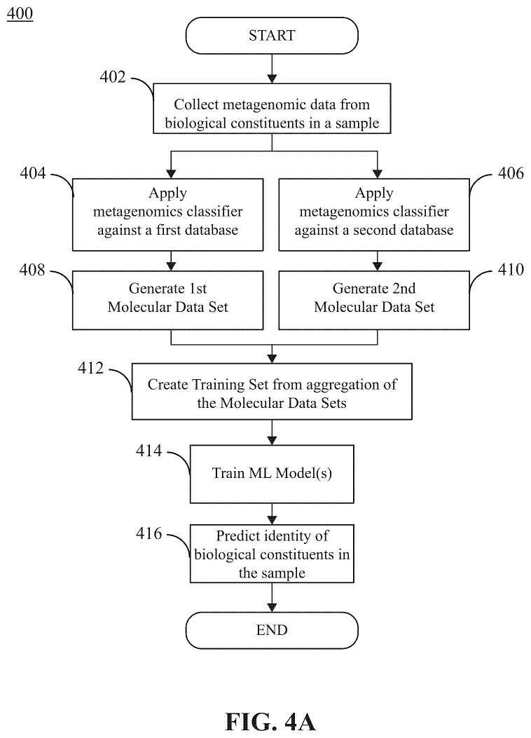

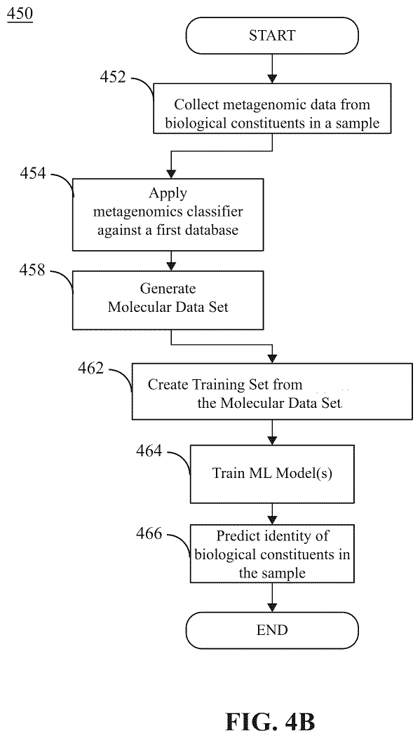

FIG. 4A shows a computer-implemented method 400 of training a machine learning model for detecting biological constituents in a sample. The method includes collecting metagenomic data from biological constituents in a sample as shown in block 402. The method further includes applying a metagenomics classifier on the collected biological constituents from the sample against a first database as shown in block 404. The method then moves to a block 408 to generate the first molecular data set. In some embodiments, the first database includes curated sequence data. In some embodiments, the curated sequence data includes polynucleotide sequences. In some embodiments, the curated sequence database includes polypeptide sequences.

After the method collects metagenomic data from the sample at the block 402 the method also moves to a block 406 wherein a metagenomic classifier is applied on the collected biological constituents from the sample against a second database. The method then moves to a block 410 to generate the second molecular data set. In some embodiments, the second database includes a publicly available sequence data. In some embodiments, the publicly available sequence data includes polynucleotides. In some embodiments, the publicly available sequence data includes polynucleotides. Once the first and second molecular data sets have been created, the method includes creating a training set, the training set including an aggregated set of the first and second molecular data sets at a block 412. Based on the output provided by the trained ML model, the method moves to a block 414 to train the ML models. Based on the output provided by the trained ML model, the method moves to a block 416 to predict the identity of biological constituents from the sample. In some embodiments, based on the output provided by the trained ML model, a prediction of an absence of the biological constituents from the sample is provided. In some embodiments, the first and second molecular data sets are produced iteratively, which further updates the training set thereby continuing to train the ML model(s).

In some embodiments, the machine learning model includes a random forest model. In some embodiments, the machine learning model includes a deep neural network (DNN). In some embodiments, the machine learning model includes a convolutional neural network (CNN). In some embodiments, the machine learning model includes a support vector machine. In some embodiments, the machine learning model includes a categorical classifier. In some embodiments, the method further includes selecting a first machine learning model based on one or more metrics of the first molecular data set. In some embodiments, the method further includes selecting a second machine learning model based on one or more metrics of the second molecular data set. In some embodiments, the metrics used for the selection of the machine learning model includes the abundance of data available to train on, relative difficulty for producing a model for a particular organism. In some embodiments, a type of machine learning model is the same for each molecular data set. In some embodiments, the type of machine learning model is different for each molecular data set. In some embodiments, the type of machine learning model is same for some of the molecular data sets and different for other molecular data sets in a plurality of molecular data sets. In some embodiments, different types of ML models could be used for different organisms.

In some embodiments, the generating of the first molecular data set includes applying an aligner-based classifier to the collected metagenomic data against a first data source, and the generating of the second molecular data set includes applying the aligner-based classifier to the collected metagenomic data against a second data source. In some embodiments, the generating of the first molecular data set includes applying a de novo assembler to the collected metagenomic data against a first data source, and the generating of the second molecular data set includes applying the de novo assembler to the collected metagenomic data against a second data source. In some embodiments, the generating of the first molecular data set includes applying a k-mer based classifier to the collected metagenomic data against a first data source, and the generating of the second molecular data set includes applying the k-mer based classifier to the collected metagenomic data against a second data source. In some embodiments, the generating of the first molecular data set includes applying a classifier to the collected metagenomic data against a first data source, and the generating of the second molecular data set includes applying the classifier to the collected metagenomic data against a second data source. In some embodiments, the classifier includes a categorical classifier. In some embodiments, the classifier includes a k-mer based classifier. In some embodiments, the classifier applied on the first molecular data set is different from the classifier applied on the second molecular data set. In some embodiments, the classifier applied on the first molecular data set is the same classifier applied on the second molecular data set.

In some embodiments, the first and second molecular data sets include polypeptides. In some embodiments, the first and second molecular data sets include polynucleotides. In some embodiments, the first data source includes a curated set of polynucleotides. In some embodiments, the curated set of polynucleotides includes one or more genomes. In some embodiments, the polynucleotides of the second molecular data set include publicly available polynucleotides. In some embodiments, the publicly available polynucleotides include one or more publicly available genomes. In some embodiments, the first data source includes a curated set of polypeptides. In some embodiments, the curated set of the polypeptides includes one or more proteomes. In some embodiments, the second data source includes publicly available polypeptides. In some embodiments, the publicly available polypeptides include one or more publicly available proteomes.

FIG. 4B shows another computer-implemented method 450 of training a machine learning model for detecting biological constituents in a sample. The method includes collecting metagenomic data from biological constituents in a sample as shown in block 452. The method further includes applying a metagenomics classifier on the collected biological constituents from the sample against a database as shown in block 454. The method then moves to a block 458 to generate the molecular data set. In some embodiments, the database includes curated sequence data. In some embodiments, the curated sequence data includes polynucleotide sequences. In some embodiments, the curated sequence database includes polypeptide sequences. In some embodiments, the database includes a publicly available sequence data. In some embodiments, the publicly available sequence data includes polynucleotides.

In some embodiments, the publicly available sequence data includes polynucleotides. Once the first and second molecular data set has been created, the method includes creating a training set at a block 462. Based on the output provided by the trained ML model, the method moves to a block 464 to train the ML models. Based on the output provided by the trained ML model, the method moves to a block 466 to predict the identity of biological constituents from the sample. In some embodiments, based on the output provided by the trained ML model, a prediction of an absence of the biological constituents from the sample is provided. In some embodiments, the molecular data set is produced iteratively, which further updates the training set thereby continuing to train the ML model(s).

In some embodiments, the machine learning model includes a random forest model. In some embodiments, the machine learning model includes a deep neural network (DNN). In some embodiments, the machine learning model includes a convolutional neural network (CNN). In some embodiments, the machine learning model includes a support vector machine. In some embodiments, the machine learning model includes a categorical classifier.

In some embodiments, the generating of the molecular data set includes applying an aligner-based classifier to the collected metagenomic data against a data source. In some embodiments, the generating of the molecular data set includes applying a de novo assembler to the collected metagenomic data against a data source. In some embodiments, the generating of the molecular data set includes applying a k-mer based classifier to the collected metagenomic data against a data source. In some embodiments, the generating of the molecular data set includes applying a classifier to the collected metagenomic data against a data source. In some embodiments, the classifier includes a categorical classifier. In some embodiments, the classifier includes a k-mer based classifier.

In some embodiments, the molecular data set includes polypeptides. In some embodiments, the molecular data set includes polynucleotides. In some embodiments, the data source includes a curated set of polynucleotides. In some embodiments, the curated set of polynucleotides includes one or more genomes. In some embodiments, the molecular data set includes publicly available polynucleotides. In some embodiments, the publicly available polynucleotides include one or more publicly available genomes. In some embodiments, the data source includes a curated set of polypeptides. In some embodiments, the curated set of the polypeptides includes one or more proteomes. In some embodiments, the data source includes publicly available polypeptides. In some embodiments, the publicly available polypeptides include one or more publicly available proteomes.

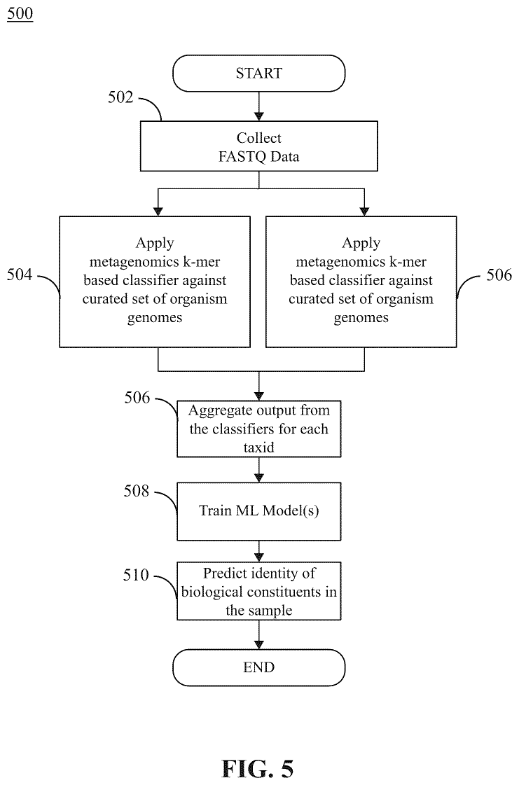

FIG. 5 shows a computer-implemented method 500 of training a machine learning model for detecting biological constituents in a sample. The method including collecting metagenomic data in block 502 from a biological constituents in a sample. The method further includes generating a first k-mer data set by applying a k-mer based classifier to the metagenomic data against a curated set of organism genomes at a block 504, generating a second k-mer data set by applying the k-mer based classifier to the metagenomic data against a subset of nucleotides from a private or publicly available database (e.g., a subset of NCBI blast database) at a block 506; aggregating the output from the classifiers for each taxon identity (taxid) at a block 506 and training the machine learning model using the training set at a block 508. Based on the output provided by the trained ML model, the method moves to a block 510 to predict the identity of biological constituents from the sample. In some embodiments, the number of molecular data sets is greater than two. In some embodiments, the number of molecular data sets is 3, 4, 5, 6, 7, 8, 9, 10, 50, 100, 200, 500, 1000, 2500, 5000, 10000, 50000, 100000, 500000, 1000000, or more, or a number in a range defined by any two of the preceding values. As shown in FIG. 5, to build machine learning models that predict the present of a species of nucleic acids in a sample, the read data may be processed through a bioinformatics pipeline towards building a training set to train the machine learning model. In some embodiments, the first and second k-mer data sets include a plurality of taxids. In some embodiments, the method further includes aggregating the first and second k-mer data sets for each of the taxids. In some embodiments, the k-mer based classifier includes or extends Taxonomer functionality. Taxonomer is discussed in U.S. Pat. No. 11,335,436, which is incorporated by reference in its entirety herein. In some embodiments, the k-mer based classifier includes KRAKEN (genomebiology.biomedcentral.com/articles/10.1186/s13059-016-0969-1).

In some embodiments, samples from which polynucleotides may be derived for analysis by the present methods and systems can be from any of a variety of sources. Non-limiting examples of sample sources include environmental sources, industrial sources, one or more subjects, and one or more populations of microbes. Examples of environmental sources include, but are not limited to agricultural fields, soils, dirt, lakes, rivers, oceans, water reservoirs, air vents, walls, roofs, soil samples, plants, and swimming pools. Examples of industrial sources include, but are not limited to clean rooms, hospitals, food processing areas, food production areas, food stuffs, medical laboratories, pharmacies, pharmaceutical compounding centers, and wastewater treatment plants. Biological constituents may be isolated from chromalveolata, such as malaria, and dinoflagellates. Non-limiting examples of subjects from which biological constituents may be isolated include multicellular organisms, such as fish, amphibians, reptiles, birds, and mammals. Non-limiting examples of mammals include be primates (e.g., apes, monkeys, gorillas), rodents (e.g., mice, rats), farm animals (e.g., cows, pigs, sheep, horses), dogs, cats, or rabbits. In some embodiments, the mammal is a human. In some embodiments, the mammal is an individual subject. A sample may include a sample from a subject, such as biological fluid; whole blood; blood products; red blood cells; white blood cells; buffy coat; swabs; urine; sputum; saliva; nasal discharge; mucus; semen; lymphatic fluid; amniotic fluid; cerebrospinal fluid; peritoneal effusions; pleural effusions; biopsy samples; fluid from cysts; synovial fluid; vitreous humor; aqueous humor; bursa fluid; eye washes; eye aspirates; plasma; serum; pulmonary lavage; lung aspirates; animal, including human, tissues, including but not limited to, liver, spleen, kidney, lung, intestine, brain, heart, muscle, pancreas, cell cultures, as well as lysates, extracts, or materials and fractions obtained from the samples described above or any cells and microbes and viruses that may be present on or in a sample. Tissues, cells, and their progeny of a biological entity obtained in vivo or cultured in vitro are also encompassed.

A sample may include cells of a primary culture or a cell line. Examples of cell lines include, but are not limited to 293-T human kidney cells, A2870 human ovary cells, A431 human epithelium, B35 rat neuroblastoma cells, BHK-21 hamster kidney cells, BR293 human breast cells, CHO chinese hamster ovary cells, CORL23 human lung cells, HeLa cells, or Jurkat cells. The sample may include a homogeneous or mixed population of microbes, including one or more of viruses, bacteria, protists, monerans, chromalveolata, archaca, or fungi. In some embodiments, the microbes are pathogens. In some embodiments, the microbes are human pathogens. Examples of viruses include, but are not limited to, ebola virus, hepatitis virus, herpesviruses, human immunodeficiency virus, influenza, lettuce big-vein associated virus, mosaic viruses, rhinovirus, ringspot virus, rotavirus, West Nile virus. Examples of bacteria include, but are not limited to, Bacillus cereus, Citrobacter koseri, Clostridium perfringens, E. coli, Enterobacter aerogenes, Enterococcus faecalis, K. pneumonia, Lactobacillus acidophilus, Listeria monocytogenes, Micrococcus luteus, Propionibacterium granulosum, Pseudomonas aeruginosa, Serratia marcescens, Staphylococcus aureus, Staphylococcus aureus Mu3, Staphylococcus aureus Mu50, Staphylococcus epidermidis, Staphylococcus simulans, Streptococcus agalactiae, Streptococcus pneumonia, Streptococcus pyogenes, and Yersinia enterocolitica. Examples of fungi include, but are not limited to, Absidia corymbifera, Aspergillus niger, Aspergillus niger, Candida albicans, Candida albicans, Geotrichum candidum, Geotrichum candidum, Hansenula anomala, Hansenula anomala, Microsporum gypseum, Microsporum gypseum, Monilia, Monilia spp., Mucor, Mucor spp., Penicillium expansum, Penicilliusidia corymbifera, Rhizopus, Rhodotorula, Saccharomyces bayabus, Saccharomyces carlsbergensis, Saccharomyces cerivisiae, and Saccharomyces uvarum. A sample can also be processed samples, such as preserved, fixed and/or stabilized samples. A sample can include or consist essentially of polypeptides. A sample can include or consist essentially of polynucleotides. A sample can include or consist essentially of RNA. A sample can include or consist essentially of DNA. In some embodiments, cell-free polynucleotides (e.g., cell-free DNA and/or cell-free RNA) are analyzed. In general, cell-free polynucleotides are extracellular polynucleotides present in a sample (e.g., a sample from which cells have been removed, a sample that is not subjected to a lysis step, or a sample that is treated to separate cellular polynucleotides from extracellular polynucleotides). In a non-limiting example, cell-free polynucleotides include polynucleotides released into circulation upon death of a cell and are isolated as cell-free polynucleotides from the plasma fraction of a blood sample.

Methods for the extraction and purification of nucleic acids are well known in the art. For example, nucleic acids can be purified by organic extraction with phenol, phenol/chloroform/isoamyl alcohol, or similar formulations, including TRIzol and TriReagent. Other non-limiting examples of extraction techniques include: (1) organic extraction followed by ethanol precipitation, e.g., using a phenol/chloroform organic reagent with or without the use of an automated nucleic acid extractor, e.g., the Model 341 DNA Extractor available from Applied Biosystems (Foster City, Calif.); (2) stationary phase adsorption methods; and (3) salt-induced nucleic acid precipitation methods, such precipitation methods being typically referred to as “salting-out” methods. Another example of nucleic acid isolation and/or purification includes the use of magnetic particles to which nucleic acids can specifically or non-specifically bind, followed by isolation of the beads using a magnet, and washing and eluting the nucleic acids from the beads. In some embodiments, the above isolation methods may be preceded by an enzyme digestion step to help eliminate unwanted protein from the sample, e.g., digestion with proteinase K, or other like proteases. If desired, Rnase inhibitors may be added to the lysis buffer. For certain cell or sample types, it may be desirable to add a protein denaturation/digestion step to the protocol. Purification methods may be directed to isolate DNA, RNA, or both. When both DNA and RNA are isolated together during or subsequent to an extraction procedure, further steps may be employed to purify one or both separately from the other. Sub-fractions of extracted nucleic acids can also be generated, for example, purification by size, sequence, or other physical or chemical.

In some embodiments, the sample may be used directly as obtained from one or more sources. In some embodiments the sample, may be pretreated to modify the character of the sample. In some embodiments, such pretreatment may include preparing plasma from blood, diluting viscous fluids and so forth. In some embodiments, methods of pretreatment may also involve, but are not limited to, filtration, precipitation, dilution, distillation, mixing, centrifugation, freezing, lyophilization, concentration, amplification, nucleic acid fragmentation, inactivation of interfering components, the addition of reagents, lysing, etc. If such methods of pretreatment are employed with respect to the sample, such pretreatment methods are typically such that the nucleic acid(s) of interest remain in the test sample, sometimes at a concentration proportional to that in an untreated test sample (e.g., namely, a sample that is not subjected to any such pretreatment method(s)).

In some embodiments, each sample structured data file is mapped against two or more separate databases. In some embodiments, the structured data file includes a FASTQ file. In some embodiments, a first database includes the first set of polynucleotides. In some embodiments, a second database includes the second set of polynucleotides. In some embodiments, the first set of polynucleotides includes a curated set of polynucleotides. In some embodiments, the curated set of polynucleotides includes one or more genomes. In some embodiments, the curated set of polynucleotides includes one or more nucleotide sequences. In some embodiments, the nucleotide sequences correspond to one or more taxa. In some embodiments, at least 1, 2, 5, 10, 25, 50, 50, 100, 250, 500, 1000, 5000, 10000, 50000, 100000, 250000, 500000, 1000000, or more, or a number in a range defined by any two of the preceding values, different taxa are identified as absent or present (and optionally abundance, which may be relative) based on sequences analyzed by any method described herein. In some embodiments, the second set of polynucleotides includes publicly available polynucleotides. In some embodiments, the publicly available polynucleotides include one or more publicly available genomes. Exemplary databases that provide the publicly available polynucleotides include the Sequence Read Archive (SRA) database, which is the largest publicly available public repository of high-throughput sequencing data. The National Center for Biotechnology Information (NCBI) hosts the SRA database, which includes over 51 quadrillion total bases.

In some embodiments, the generating of the first and second data sets occurs in parallel. In some embodiments, the generating of the data sets occurs sequentially. In some embodiments, the generating of the data sets occurs on a schedule. In some embodiments, more than two data sets are generated. In some embodiments, the method further includes detecting, from an output of the machine learning model using the training set, a species of the polynucleotides obtained from the sample. In some embodiments, the machine learning model includes a random forest (RF) model. In some embodiments, the machine learning model includes a deep neural network (DNN). In some embodiments, the machine learning model includes a support vector machine (SVN). In some embodiments, the machine learning model includes a convolution neural network (CNN). In some embodiments, the machine learning model includes a categorical classifier.

In some embodiments, molecular attributes or covariates are extracted from each molecular dataset and put into a table where the covariates can be used for metagenomics model training/testing. The metagenomics k-mer based classifier may use a HyperLogLog (en.wikipedia.org/wiki/HyperLogLog) data structure to keep track of the number of distinct k-mers from the reads assigned to a taxid. This data structure provides how much distinct data (as opposed to a single repeated sequence) is assigned to a taxid. The 23etagenomics classifier may also compute this value for each taxid based on the reference sequences when the reference index is being computed (prior to any classification). With this data, it possible to ascertain the percentage of distinct k-mers that occur in the sample compared to a reference database.

In some embodiments, metagenomics data covariates are collected at a taxid level. In some embodiments, the covariates include read count (from the first and second databases), intersection of read identifiers classifying to the same taxid from the different databases, distinct k-mer count, median classification read score, or a combination thereof. In the machine learning model that uses an RF model, covariate importance is computed when the RF model is built. A non-limiting example of covariate importance is provided in Table 1 shown below.

TABLE 1
Non-limiting example of feature importance assigned when
constructing an RF model using XGBoost

| Covariates | Importance |
|---|---|
| Database 1 read count | 0.03623812 |
| Database 2 read count | 0.01373014 |
| Both databases read count | 0.01377384 |
| Distinct k-mer count | 0.83370483 |
| Median classification read score | 0.09675267 |
| Median NT score | 0.00580038 |

In some embodiments, the method further includes detecting, from an output of the machine learning model using the training set, a presence of biological constituents obtained from the sample. In some embodiments, the presence of the biological constituents is provided as a probability value from an output of the machine learning trained on the training set. In some embodiments, the probability value is taken at face value such that a probability value of above 0.5 for a predicted/identified biological constituent is indicative of presence. In some embodiments, the probability value is greater than 0.5 depending on the biological constituent. In some embodiments, the probability value is adjusted based on the training set. In some embodiments, the training set improves upon an older training set. In some embodiments, the method further includes detecting, from an output of the machine learning model using the training set, an absence of a biological constituent obtained from the sample. In some embodiments, the detecting further includes detecting, from the output of the machine learning model using the training set, an absence of the biological constituent obtained from the sample. In some embodiments, the sample is sourced from one or more environmental sources, one or more industrial sources, one or more subjects, one or more populations of microbes, or a combination thereof. In some embodiments, the biological constituents obtained from the sample includes biological constituents from one or more pathogens.

In some embodiments, a system for detecting biological constituents in a sample is provided, the system including a computer readable medium storing instructions, and one or more processors programmed to read the instructions to execute the method of any of the embodiments provided herein.

In practice, specific model choice is not as important as being able to control overfitting given the available data in the training set and having the model appropriately generalize. Desirously, RF models in the XGBoost framework provide enough parameters to tune models to protect against overfitting even with relatively small amounts of data in the training set. Another desirous property of using RF models from XGBoost is the ability to perform online learning. As more data is processed, with the appropriate labeling, machine learning models can be updated “online” such that the machine learning models do not have to be rebuilt whenever they are trained with new data. As used herein “online learning,” interchangeable with “incremental learning” and “out-of-core learning,” is a machine learning paradigm where the model updates its knowledge incrementally as new data instances become available, instead of processing the entire dataset in a batch fashion. This approach is particularly useful when handling large-scale data sets that cannot fit into memory or when data is received in a continuous stream.

### EXAMPLE 1

To build training sets for the machine learning model, a plurality of sequence data sets that are known to have signal for microbes from a variety of sources are used for the training sets, the sources including public database sources, environmental sources, industrial sources, subjects, or a combination. The machine learning model is trained on such training sets where the microbe is known. This approach is iterated for multiple known microbes. After the machine learning model is trained from the plurality of training sets, samples that may include microbes of unknown identity are processed as shown in FIG. 5, whereby the sample is processed and the nucleic acids in the sample, if any, are sequenced to obtain sequence data.

For this example, the sequence data is stored in a FASTQ format. Alternative formats may be used. The sequence data is then transformed by k-mer-based classifiers and/or aligners and/or assemblers to produce labeled data about the microbes that may be present in the sample. This labeled data forms at least part of the training set that is used to train the machine learning model. When the machine learning model is trained against a training set that includes data sets for various human pathogens, subsequent prediction of human pathogens in samples using the trained models displays increased accuracy and sensitivity compared to on-market metagenomics pipelines that use various standard metrics and set thresholds.

A single model for every organism is often ineffective. What is effective is building multiple models for individual organisms because different organisms have different genomic landscapes, they also exist in different communities and likely have more closely related neighbors than less, for example microbes. There are many genomic factors that go into having more than one model per organism.

The number of different data sets of a particular organism needed to obtain a training set for training the machine learning model to make accurate predictions vary with the organism depending on the comparative genomic landscape. Generally, at least 20 or more positive samples for a particular organism is sufficient for the machine learning model to produce accurate predictions of microbes, e.g., identities of the microbes. However, some pathogens, such as E. coli, need at least 100 or more positive samples to effectively distinguish the signal and noise from other microbes that may be present in a sample that is to be tested. This higher count is due to the potential of many species of nucleic acids being present in the sample and how E. coli is similar to other microbes in the same family that co-occur in samples, so distinguishing these differences is important.

### EXAMPLE 2

To build machine learning models to aid in the detection of organisms, multiple sequencing data sets may be used. These data sets often fall into one of two categories: 1. Simulated data sets where reads are generated in-silico and the contents are completely known and controlled; 2. Real sequencing experiments derived from known samples or from samples that have had their sequenced contents characterized. The sequencing contents from the real sequencing experiments often contain unexpected reads for reasons, such as sequencing reagent contamination or other unexpected organisms present in the sample environment.

Sequencing data sets from real samples are often more effective for training machine learning models than their simulated counterparts for detecting microbes because the data contains noise and organism genome variation found in real samples. However, for practical reasons, it can be difficult to build a training set that consists only of real sequencing experiments. Moreover, simulated data sets are typically needed to augment the training set in order to provide a complete and robust ML model.

Multiple sequencing data sets are normally used for each organism to be detected by the model(s). Generally, at least 20 positive data sets for each organism (usually a mix of real and simulated samples) are used to effectively train the model; more training data typically leads to a better model. Some organisms need more training data than others to effectively distinguish signal from noise. For example, Organism A may need 200 samples to produce accurate predictions while another may need only 10 because organisms have different genomic landscapes, exist in different communities, and have varying levels of genetic relatedness to others. For these reasons, a single model applied to all organisms is usually not as effective as building a separate model for each organism.

The training data sets are processed by sending each sample through a k-mer classifier to be classified against a set of reference sequences. In FIG. 5, this would mean each sample in the training dataset would be classified a least twice, once for each set of reference sequences. For each sample, the classifier output is combined from the two different classifications (one from each database or collection of references) to create the aggregate covariates that are used to train the model. The combination of data classified against the different reference collections is then used to train the organism models.

### EXAMPLE 3

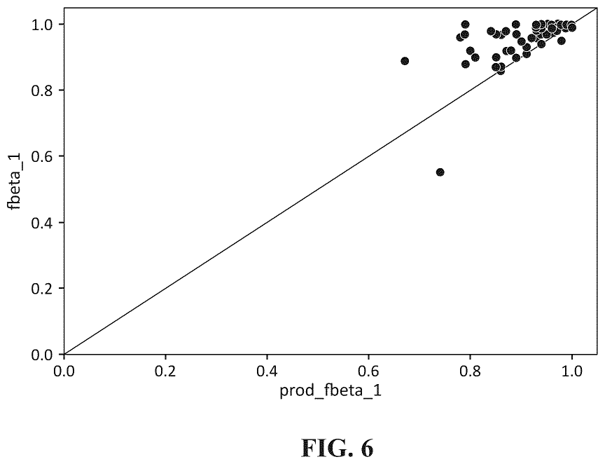

Using genome simulations and read data from the Sequence Read Archive (SRA), test data was generated using RF models in the pipeline shown in FIG. 5 to train and test models for about 100 or more different bacterial uropathogens. For comparison, the same test data was processed using the Explify Urinary Pathogen ID/AMR Panel (UPIP) Data Analysis application available in Illumina BaseSpace Sequence Hub (BSSH) to call which uropathogens are present in each sample. FIG. 6 compares the results from RF models built using the XGBoost framework to call uropathogens in a test set of genome simulatinons and SRA data to the results from the Explify UPIP pipeline. Using F beta score (harmonic mean between recall and precision), the pipeline using machine learning based on RF models (y-axis) outperformed the Explify UPIP pipeline (x-axis) overall.

### EXAMPLE 4

As discussed above, machine learning models can be used to dramatically increase organism detection accuracy with k-mer based classifiers. To demonstrate this, a simulation study with k-mer classifiers using simulated reads, and a collection of 27,155 bacterial 16S rRNA sequences that were gathered from the NCBI in April 2024, was performed. Read classification and organism detection with a k-mer classifier using this set of 16S references is challenging because the conserved nature of the 16S gene means that there is relatively little genetic difference between 16S genes of different species.

The simulation study was setup as follows: 1000 distinct taxa represented in the gathered 16S sequences were randomly selected. Then for each of these taxa, one of the corresponding 16S sequences was selected to be a simulated reference. For taxa that contain more than one 16S sequence, the simulation candidate was removed from the larger collection to challenge the classifiers with a sequence not directly represented in the database. For taxa that contain just a single reference 16S sequence, it was left in the reference collection to ensure the taxa is represented during classification. After these 1000 references for simulation were selected and the reference collection was updated, reference indices for two different tools, DRAGEN® k-mer classifier and KRAKEN2® classifier, were constructed. For each selected reference for simulation, the NCBI's ART simulator was used to simulate paired-end 150 bp reads at 20× read depth with an Illumina HiSeq® error profile, similar to block 452 of FIG. 4B. The reads for each of the 1000 simulated references were then classified using either the DRAGEN® k-mer classifier or the KRAKEN2® classifier, according to block 454 of FIG. 4B. The classification results were aggregated into a table, according to block 458 of FIG. 4B, so that recall, precision, and accuracy can be generated.

Once the simulated data is aggregated, machine learning models using the XGBoost framework were constructed using 6 of the features produced in columns of the table by the DRAGEN k-mer classifier, according to block 462 of FIG. 4B. The 6 features were duplicity, distinct_coverage, read_count, total_kmer_count, distinct_kmer_count, taxid_distinct_kmer_count. As for the expected outcome feature, the information about which reference was simulated was used to label each row of the table as taxa being expected to be present with a ‘1’, and with a ‘0’ if otherwise. A classification model was trained, according to block 464 of FIG. 4B, using the XGBoost framework with a random subset of the aggregated simulation data, and the trained classification model was tested using an independent random subset of the same simulation data to demonstrate the utility of machine learning using the above features to increase organism taxa accuracy. Table 2 shows the results of this study. As shown in Table 2, combining machine learning and the DRAGEN® classifier enhances the performance significantly compared to using either the DRAGEN® or the KRAKEN2® classifier alone.

TABLE 2
Results of the simulation study described in EXAMPLE 4

| Tool | Recall | Precision | F1-score |
|---|---|---|---|
| DRAGEN | .75 | .66 | .58 |
| KRAKEN2 | .74 | .65 | .57 |
| DRAGEN + ML | .93 | .93 | .93 |

### Definitions

The section headings used herein are for organizational purposes only and are not to be construed as limiting the subject matter described.

Unless defined otherwise, all technical and scientific terms used herein have the same meaning as is commonly understood by one of ordinary skill in the art. The use of the term “including” as well as other forms, such as “include”, “includes,” and “included,” is not limiting. The use of the term “having” as well as other forms, such as “have”, “has,” and “had,” is not limiting. As used in this specification, whether in a transitional phrase or in the body of the claim, the terms “comprise(s)” and “comprising” are to be interpreted as having an open-ended meaning. That is, the above terms are to be interpreted synonymously with the phrases “having at least” or “including at least.” For example, when used in the context of a process, the term “comprising” means that the process includes at least the recited steps, but may include additional steps. When used in the context of a compound, composition, or device, the term “comprising” means that the compound, composition, or device includes at least the recited features or components, but may also include additional features or components.

The terms “polynucleotide,” “oligonucleotide,” “nucleic acid” and “nucleic acid molecules” are used interchangeably herein and refer to a covalently linked sequence of nucleotides of any length (i.e., ribonucleotides for RNA, deoxyribonucleotides for DNA, analogs thereof, or mixtures thereof) in which the 3′ position of the pentose of one nucleotide is joined by a phosphodiester group to the 5′ position of the pentose of the next. The terms should be understood to include, as equivalents, analogs of either DNA, RNA, cDNA, or antibody-oligo conjugates made from nucleotide analogs and to be applicable to single stranded (such as sense or antisense) and double stranded polynucleotides. The term as used herein also encompasses cDNA, that is complementary or copy DNA produced from an RNA template, for example by the action of reverse transcriptase. This term refers only to the primary structure of the molecule. Thus, the term includes, without limitation, triple-, double-and single-stranded deoxyribonucleic acid (“DNA”), as well as triple-, double-and single-stranded ribonucleic acid (“RNA”). The nucleotides include sequences of any form of nucleic acid.

### Additional Notes

Various embodiments of the present disclosure may be a system, a method, and/or a computer program product at any possible technical detail level of integration. The computer program product may include a computer readable storage medium (or mediums) having computer readable program instructions thereon for causing a processor to carry out aspects of the present disclosure.

For example, the functionality described herein may be performed as software instructions are executed by, and/or in response to software instructions being executed by, one or more hardware processors and/or any other suitable computing devices. The software instructions and/or other executable code may be read from a computer readable storage medium (or mediums). Computer readable storage mediums may also be referred to herein as computer readable storage or computer readable storage devices.

The computer readable storage medium can be a tangible device that can retain and store data and/or instructions for use by an instruction execution device. The computer readable storage medium may be, for example, but is not limited to, an electronic storage device (including any volatile and/or non-volatile electronic storage devices), a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a solid state drive, a random access memory (RAM), a read-only memory (ROM), an crasable programmable read-only memory (EPROM or Flash memory), a static random access memory (SRAM), a portable compact disc read-only memory (CD-ROM), a digital versatile disk (DVD), a memory stick, a floppy disk, a mechanically encoded device, such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers, and/or edge servers. A network adapter card or network interface in each computing/processing device receives computer readable program instructions from the network and forwards the computer readable program instructions for storage in a computer readable storage medium within the respective computing/processing device.

Computer readable program instructions (as also referred to herein as, for example, “code,” “instructions,” “module,” “application,” “software application,” and/or the like) for carrying out operations of the present disclosure may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language, such as Smalltalk, C++, or the like, and procedural programming languages, such as the “C” programming language or similar programming languages. Computer readable program instructions may be callable from other instructions or from itself, and/or may be invoked in response to detected events or interrupts. Computer readable program instructions configured for execution on computing devices may be provided on a computer readable storage medium, and/or as a digital download (and may be originally stored in a compressed or installable format that requires installation, decompression or decryption prior to execution) that may then be stored on a computer readable storage medium. Such computer readable program instructions may be stored, partially or fully, on a memory device (e.g., a computer readable storage medium) of the executing computing device, for execution by the computing device. The computer readable program instructions may execute entirely on a user's computer (e.g., the executing computing device), partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present disclosure.

Aspects of the present disclosure are described herein with reference to flowchart illustrations and/or block diagrams of methods, apparatus (systems), and computer program products according to embodiments of the disclosure. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart(s) and/or block diagram(s) block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks. For example, the instructions may initially be carried on a magnetic disk or solid-state drive of a remote computer. The remote computer may load the instructions and/or modules into its dynamic memory and send the instructions over a telephone, cable, or optical line using a modem. A modem local to a server computing system may receive the data on the telephone/cable/optical line and use a converter device including the appropriate circuitry to place the data on a bus. The bus may carry the data to a memory, from which a processor may retrieve and execute the instructions. The instructions received by the memory may optionally be stored on a storage device (e.g., a solid-state drive) either before or after execution by the computer processor.

The flowchart and block diagrams in the Figures illustrate the architecture, functionality, and operation of possible implementations of systems, methods, and computer program products according to various embodiments of the present disclosure. In this regard, cach block in the flowchart or block diagrams may represent a service, module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. In addition, certain blocks may be omitted in some implementations. The methods and processes described herein are also not limited to any particular sequence, and the blocks or states relating thereto can be performed in other sequences that are appropriate.

It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions. For example, any of the processes, methods, algorithms, clements, blocks, applications, or other functionality (or portions of functionality) described in the preceding sections may be embodied in, and/or fully or partially automated via, electronic hardware such application-specific processors (e.g., application-specific integrated circuits (ASICs)), programmable processors (e.g., field programmable gate arrays (FPGAs)), application-specific circuitry, and/or the like (any of which may also combine custom hard-wired logic, logic circuits, ASICs, FPGAs, etc. with custom programming/execution of software instructions to accomplish the techniques).

Any of the above-mentioned processors, and/or devices incorporating any of the above-mentioned processors, may be referred to herein as, for example, “computers,” “computer devices,” “computing devices,” “hardware computing devices,” “hardware processors,” “processing units,” and/or the like. Computing devices of the above-embodiments may generally (but not necessarily) be controlled and/or coordinated by operating system software, such as Mac OS, IOS, Android, Chrome OS, Windows OS (e.g., Windows XP, Windows Vista, Windows 7, Windows 8, Windows 10, Windows 11, Windows Server, etc.), Windows CE, Unix, Linux, SunOS, Solaris, Blackberry OS, VxWorks, or other suitable operating systems. In other embodiments, the computing devices may be controlled by a proprietary operating system. Conventional operating systems control and schedule computer processes for execution, perform memory management, provide file system, networking, I/O services, and provide a user interface functionality, such as a graphical user interface (“GUI”), among other things.

Reference throughout the specification to “one example”, “another example”, “an example”, and so forth, means that a particular element (e.g., feature, structure, and/or characteristic) described in connection with the example is included in at least one example described herein, and may or may not be present in other examples. In addition, it is to be understood that the described elements for any example may be combined in any suitable manner in the various examples unless the context clearly dictates otherwise.

It is to be understood that the ranges provided herein include the stated range and any value or sub-range within the stated range, as if such value or sub-range were explicitly recited. For example, a range from about 2 kbp to about 20 kbp should be interpreted to include not only the explicitly recited limits of from about 2 kbp to about 20 kbp, but also to include individual values, such as about 3.5 kbp, about 8 kbp, about 18.2 kbp, etc., and sub-ranges, such as from about 5 kbp to about 10 kbp, etc. Furthermore, when “about” and/or “substantially” are/is utilized to describe a value, this is meant to encompass minor variations (up to +/−10%) from the stated value.

While several examples have been described in detail, it is to be understood that the disclosed examples may be modified. Therefore, the foregoing description is to be considered non-limiting.

While certain examples have been described, these examples have been presented by way of example only, and are not intended to limit the scope of the disclosure. Indeed, the novel methods described herein may be embodied in a variety of other forms. Furthermore, various omissions, substitutions and changes in the methods described herein may be made without departing from the spirit of the disclosure. The accompanying claims and their equivalents are intended to cover such forms or modifications as would fall within the scope and spirit of the disclosure.

Features, materials, characteristics, or groups described in conjunction with a particular aspect, or example are to be understood to be applicable to any other aspect or example described in this section or elsewhere in this specification unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing examples. The protection extends to any novel one, or any novel combination, of the features disclosed in this specification (including any accompanying claims, abstract and drawings), or to any novel one, or any novel combination, of the steps of any method or process so disclosed.

Furthermore, certain features that are described in this disclosure in the context of separate implementations can also be implemented in combination in a single implementation. Conversely, various features that are described in the context of a single implementation can also be implemented in multiple implementations separately or in any suitable sub-combination. Moreover, although features may be described above as acting in certain combinations, one or more features from a claimed combination can, in some cases, be excised from the combination, and the combination may be claimed as a sub-combination or variation of a sub-combination.

Moreover, while operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that in some examples, the actual steps taken in the processes illustrated and/or disclosed may differ from those shown in the figures. Depending on the example, certain of the steps described above may be removed or others may be added. Furthermore, the features and attributes of the specific examples disclosed above may be combined in different ways to form additional examples, all of which fall within the scope of the present disclosure.

For purposes of this disclosure, certain aspects, advantages, and novel features are described herein. Not necessarily all such advantages may be achieved in accordance with any particular example. Thus, for example, those skilled in the art will recognize that the disclosure may be embodied or carried out in a manner that achieves one advantage or a group of advantages as taught herein without necessarily achieving other advantages as may be taught or suggested herein.

Conditional language, such as “can,” “could,” “might,” or “may,” unless specifically stated otherwise, or otherwise understood within the context as used, is generally intended to convey that certain examples include, while other examples do not include, certain features, elements, and/or steps. Thus, such conditional language is not generally intended to imply that features, elements, and/or steps are in any way required for one or more examples or that one or more examples necessarily include logic for deciding, with or without user input or prompting, whether these features, elements, and/or steps are included or are to be performed in any particular example.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain examples require the presence of at least one of X, at least one of Y, and at least one of Z.

Language of degree used herein, such as the terms “approximately,” “about,” “generally,” and “substantially” represent a value, amount, or characteristic close to the stated value, amount, or characteristic that still performs a desired function or achieves a desired result.

The scope of the present disclosure is not intended to be limited by the specific disclosures of preferred examples in this section or elsewhere in this specification, and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

What is claimed is:

1. A computer-implemented method of training a machine learning model for detecting biological constituents in a sample, comprising:
collecting metagenomic data from biological constituents in a sample;
generating a first molecular data set;
generating a second molecular data set;
creating a training set comprising an aggregated set of the first and second molecular data sets; and
training the machine learning model using the training set.

2. The method of claim 1, wherein the machine learning model comprises a random forest model.

3. The method of claim 1, wherein the generating of the first molecular data set comprises applying an aligner-based classifier to the collected metagenomic data against a first source, and
wherein the generating of the second molecular data set comprises applying the aligner-based classifier to the collected metagenomic data against a second source.

4. The method of claim 1, wherein the generating of the first molecular data set comprises applying a de novo assembler to the collected metagenomic data against a first source, and
wherein the generating of the second molecular data set comprises applying the de novo assembler to the collected metagenomic data against a second source.

5. The method of claim 1, wherein the generating of the first molecular data set comprises applying a k-mer based classifier to the collected metagenomic data against a first source, and
wherein the generating of the second molecular data set comprises applying the k-mer based classifier to the collected metagenomic data against a second source.

6. The method of claim 1, wherein the generating of the first molecular data set comprises applying a classifier to the collected metagenomic data against a first source, and
wherein the generating of the second molecular data set comprises applying the classifier to the collected metagenomic data against a second source.

7. The method of claim 1, wherein the first and second molecular data sets comprise a plurality of taxon identities (taxids).

8. The method of claim 1, further comprising detecting, from an output of the machine learning model using the training set, a presence of one or more of the biological constituents obtained from the sample based on a probability value.

9. The method of claim 1, further comprising detecting, from an output of the machine learning model using the training set, an absence of one or more of the biological constituents obtained from the sample.

10. The method of claim 1, wherein the sample is sourced from one or more environmental sources, one or more industrial sources, one or more subjects, one or more populations of microbes, or a combination thereof.

11. The method of claim 1, wherein the generating of the first and second molecular data sets occurs in parallel.

12. The method of claim 1, further comprising iterating the first molecular data set.

13. The method of claim 1, further comprising iterating the second molecular data set.

14. A system for detecting biological constituents in a sample, comprising:
one or more processors that are programmed to execute a method comprising:
obtaining metagenomic data, wherein the metagenomic data is obtained from biological constituents in a sample;
generating a first molecular data set;
generating a second molecular data set;
creating a training set comprising an aggregated set of the first and molecular data sets; and
training the machine learning model using the training set.

15. The system of claim 14, wherein the machine learning model comprises a random forest model.

16. The system of claim 14, wherein the generating of the first molecular data set comprises applying an aligner-based classifier to the collected metagenomic data against a first source, and
wherein the generating of the second molecular data set comprises applying the aligner-based classifier to the collected metagenomic data against a second source.

17. The system of claim 14, wherein the generating of the first molecular data set comprises applying a de novo assembler to the collected metagenomic data against a first source, and
wherein the generating of the second molecular data set comprises applying the de novo assembler to the collected metagenomic data against a second source.

18. The system of claim 14, wherein the generating of the first molecular data set comprises applying an k-mer based classifier to the collected metagenomic data against a first source, and
wherein the generating of the second molecular data set comprises applying the k-mer based classifier to the collected metagenomic data against a second source.

19. The system of claim 14, wherein the generating of the first molecular data set comprises applying a classifier to the collected metagenomic data against a first source, and
wherein the generating of the second molecular data set comprises applying the classifier to the collected metagenomic data against a second source.

20. The system of claim 14, wherein the first and second molecular data sets comprise a plurality of taxids.
